(19) United States
(12) Patent Application Publication
(10) Pub. No.: US 2005/0100939 A1
(43) Pub. Date: May 12, 2005

(54) System and methods for enhancing signal-to-noise ratios of microarray-based measurements

(76) Inventors: Eugeni Namsaraev, Sunnyvale, CA (US); George Karlin-Neumann, Palo Alto, CA (US); Malek Faham, Pacifica, CA (US); Maneesh Jain, San Francisco, CA (US); Paul Hardenbol, San Francisco, CA (US); Thomas D. Willis, San Francisco, CA (US); Zhiyong Wang, Daly City, CA (US)

Correspondence Address: STEPHEN C. MACEVICZ, 21890 RUCKER DRIVE, CUPERTINO, CA, 95014, US

(21) Appl. No.: 10/943,752

(22) Filed: Sep. 17, 2004

Related U.S. Application Data
(60) Provisional application No. 60/504,634, filed on Sep. 18, 2003.

(51) Int. Cl. C12Q 1/68; G06F 19/00; G01N 33/48; G01N 33/50

(52) U.S. Cl. 435/6; 702/20

(57) ABSTRACT

The present invention provides systems and methods for large-scale genetic measurements by generating from a sample labeled target sequences whose length, orientation, label, and degree of overlap and complementarity are tailored to corresponding end-attached probes of a solid support so that signal-to-noise ratios of measurement from specifically hybridized labeled target sequences are maximized. Systems for implementing methods of the invention include a set of sample-interacting probes to produce amplicons that either each contain a segment of a target polynucleotide or an oligonucleotide tag that corresponds to a segment of a target polynucleotide, one or more solid phase supports that contain a plurality of end-attached probes, and methods of generating from sample-interacting probe amplicons from which labeled target sequences are tailored for hybridization to the solid phase supports, such as microarrays. In one aspect, labeled target sequences and end-attached probe of the solid phase supports comprise oligonucleotide tags and tag complements, respectively, selected from a minimally cross-hybridizing set.

### CROSS-REFERENCE TO RELATED APPLICATION

This application claims benefit from U.S. provisional patent Application Ser. No. 60/504,634, filed Sep. 18, 2003, the disclosure of which is incorporated herein by reference in its entirety.

### FIELD OF THE INVENTION

The present invention relates to systems and methods for enhancing the signal-to-noise ratio of measurements of labeled target sequences hybridized to probes attached to solid phase supports, such as microarrays.

### BACKGROUND

Microarrays have been important and powerful tools for large-scale studies of gene expression, genetic variation, and the organization of the genome, e.g. Chee et al, Science, 274: 610-614 (1996); Lockhart et al, Nature Biotechnology, 14: 1675-1680 (1996); Wang et al, Science, 280: 1077-1082 (1998); Golub et al, Science, 286: 531-537 (1999); Van't Veer et al, Nature, 415: 530-536 (2002); Nature Genetics Supplement, 21: 1-60 (1999); Nature Genetics Supplement, 32: 465-552 (2002); Patil et al, Science, 294: 1719-1722 (2001); and the like. However, difficult challenges remain with the technology in a number of areas, including those related to sensitivity, e.g. the ability to detect rare target sequences or small changes in the quantities of target sequences, dynamic range, e.g. the ability to simultaneously detect target sequences of widely varying concentrations, and sample preparation and data analysis, e.g. normalization, extraction of meaningful biological information, validation, and the like, e.g. Lee, Clinical Chemistry, 47: 1350-1352 (2001); Butte, Nature Reviews Drug Discovery, 1: 951-960 (2002); Macgregor, Expert Rev. Mol. Diagn., 3: 185-200 (2003); Vacha, Agilent publication (Oct. 21, 2003).

Labeled target sequences and/or fragments are an important source of noise in microarray measurements. In most analyses, mixtures of labeled target sequences are prepared by producing labeled copies of target sequences followed by a fragmentation step that yields for each target sequence a mixture of labeled target fragments of different lengths, e.g. Hughes et al, Nature Biotechnology, 19: 342-347 (2001); Chee et al (cited above); Wang et al (cited above); Lockhart et al (cited above); Golub et al (cited above). Such procedures can lead to noise and loss of signal through cross hybridization between homologous labeled target fragments and their respective probes and through the presence of single stranded overhangs in duplexes between probes and labeled target fragments that interact with surfaces and adjacent probes to reduce duplex stability or signal intensity.

An alternative approach to the direct use of labeled target fragments involves the generation of labeled target sequences that incorporate oligonucleotide tags of defined length and sequence that are specifically hybridized to tag complements on a microarray, e.g. Brenner, U.S. Pat. No. 5,635,400; Brenner et al, Proc. Natl. Acad. Sci., 97: 1665-1670 (2000); Shoemaker et al, Nature Genetics, 14: 450-456 (1996); Morris et al, European patent publication 0799897A1; Wallace, U.S. Pat. No. 5,981,179; and the like. Generally, the oligonucleotide tags are members of minimally cross-hybridizing sets so that minimal, if any, cross hybridization occurs due to the tag moieties of the labeled target sequences. However, such labeled target sequences also generally have additional “target interacting” moieties, such as primers that are extended on target sequences, that have similar noise-generating characteristics as labeled target fragments, e.g. Fan et al, Genome Research, 10: 853-860 (2000); Chen et al, Genome Research, 10: 549-557 (2000); Hirschhorn et al, Proc. Natl. Acad. Sci., 97: 12164-12169 (2000); Fan et al, U.S. patent publication Ser. No. 2003/0003490.

The availability of microarray systems that permit measurements having improved signal-to-noise ratios would lead to improved sensitivity and dynamic range of measurements which, in turn, would lead to better large-scale analysis of a range of genetic phenomena, including gene copy number variation in health and disease, occurrence of rare variants in pooled samples, low level gene expression variation in health and disease, and the like, e.g. Albertson et al, Nature Genetics, 34: 369-376 (2003); Sebat et al, Science, 305: 525-528 (2004); and the like.

### SUMMARY OF THE INVENTION

The present invention includes systems and methods for large-scale genetic measurements by generating from a sample labeled target sequences whose length, orientation, label, and degree of overlap and complementarity are tailored to corresponding end-attached probes of a solid support so that signal-to-noise ratios of measurements from specifically hybridized labeled target sequences are maximized.

In one aspect the invention provides a method of enhancing signal-to-noise ratios of measurements from one or more solid phase supports having end-attached probes by way of the following steps: (a) providing one or more solid phase supports, each having a surface and one or more end-attached probes, each of such probes having a surface-proximal end nucleotide, a surface-distal end nucleotide, and a nucleotide sequence; (b) providing labeled target sequences from a sample such that (i) each labeled target sequence comprises a first end nucleotide, a second end nucleotide, and a nucleotide sequence complementary to the nucleotide sequence of at least one end-attached probe of a solid phase support, and (ii) in duplexes formed between labeled target sequences and end-attached probes, the first end nucleotide of each labeled target sequence overhangs the surface-proximal nucleotide of the end-attached probe by from 0 to 10, or 0 to 5, or 0 to 2 nucleotides, or is flush with such nucleotide, and the second end nucleotide of each labeled target sequence overhangs the surface-distal nucleotide of the end-attached probe by from 0 to 14, or 0 to 5, or 0 to 2 nucleotides, or is flush with such nucleotide; and (c) mixing under hybridizing conditions labeled target sequences with the one or more solid phase supports so that duplexes form between labeled target sequences and end-attached, and so that the labels of the labeled target sequences generate signals from the one or more solid phase supports.

In another aspect of the method of the invention, the one or more solid phase supports is a microarray or a random microarray each having a plurality of said end-attached probes, and the labeled target sequences comprise a set of minimally cross-hybridizing oligonucleotide tags and the end-attached probes on said microarray or said random microarray comprise a set of tag complements of such minimally cross-hybridizing oligonucleotides.

In another aspect of the method of the invention, the labeled target sequences are produced from a sample-interacting probe, which is usually a circularizing probe that has been converted into a covalently closed circle by a template-driven ligation reaction between the circularizing probe and a target nucleic acid in a sample. In a preferred embodiment, the circularizing probe is selected from the group consisting of molecular inversion probes, padlock probes, and rolling circle probes.

In still another aspect, the invention includes a method of enhancing signal-to-noise ratios of measurements from one or more solid phase supports by way of the following steps: (a) providing one or more solid phase supports, each having a surface and one or more end-attached probes, each of such probes having a surface-proximal end nucleotide, a surface-distal end nucleotide, and a nucleotide sequence; (b) providing labeled target sequences from a sample, each labeled target sequence comprising (i) a first segment having a first end nucleotide and a nucleotide sequence complementary to the nucleotide sequence of at least one end-attached and (ii) a second segment having a predetermined sequence having a length in the range of from 8 to 60 nucleotides, the second segment overhanging the surface-distal nucleotide of the end-attached probe whenever a duplex is formed between a labeled target sequence and such end-attached probe; (c) providing for each second segment one or more detection oligonucleotides, each having an end complementary to the predetermined sequence of the second segment of at least one labeled target sequence such that the end of at least one of the one or more detection oligonucleotides abuts the surface-distal nucleotide of the end-attached probe, at least one detection oligonucleotide being labeled with one or more light-generating molecules for producing optical signals or with one or more hapten molecules that may be combined with capture agents for producing optical signals; and (d) mixing under hybridizing conditions the labeled target sequences and the detection oligonucleotides with the one or more solid phase supports so that duplexes form between labeled target sequences and end-attached probes and between the second segment of labeled target sequences and detection oligonucleotides and so that the labels of the detection oligonucleotides generate signals from the one or more solid phase supports.

In one aspect, kits of the invention include one or more microarrays each having a plurality of end-attached probes, each end attached probe having a surface-proximal nucleotide and a surface-distal nucleotide; and a plurality of sample-interaction probes for generating labeled target sequences such that each labeled target sequence overhangs the surface-proximal nucleotide of a complementary end-attached probe by a number of nucleotide in the range of from 0 to 10 and the surface-distal nucleotide of a complementary end-attached probe by a number of nucleotide in the range of from 0 to 14 whenever a duplex is formed therebetween. In one aspect, said ranges are each from 0 to 2. In another aspect, sample-interacting probes of such kits are circularizing probes, in which case, kits of the invention may further include reagents for conducting template-driven ligation reactions for the purpose of forming closed covalent circles from said circularizing probes whenever a complementary target polynucleotide is present in a sample. In yet another aspect, the labeled target sequences comprises a set of minimally cross-hybridizing oligonucleotides and the end-attached probes on the microarray or random microarray comprise a set of tag complements of such minimally cross-hybridizing oligonucleotides.

In another aspect, the invention provides systems for carrying out the methods of the invention and for making genetic measurements, as described more fully below. In one aspect, genetic measurements includes the detection of single-nucleotide polymorphisms, other polymorphisms, including insertions or deletions or inversions of from 2 to 5 nucleotides, gene duplications, gene copy-number quantification, allele quantification in pooled or unpooled samples, allele frequenies, gene expression, and the like.

### BRIEF DESCRIPTION OF THE FIGURES

FIGS. 1A-1D illustrate 3′-end-attached probes and 5′-end-attached probes on solid phase supports.

FIG. 2A illustrates data of signal magnitude versus size, label position, concentration, and relative overhangs of various labeled target sequences that each comprises an identical oligonucleotide tag and that has been specifically hybridized to a microarray of end-attached probes of tag complements.

FIG. 2B illustrates the use of a circularizable probe for generating amplicons in accordance with the invention.

FIG. 3 illustrates the generation of labeled target sequences by cleavage of a labeled primer.

FIG. 4 illustrates the generation of labeled target sequences by a terminal transferase reaction.

FIG. 5 illustrates the generation of labeled target sequences by a fill-in reaction after digestion with a restriction endonuclease leaving a 5′ overhang.

FIG. 6 illustrates the generation of labeled target sequences by nuclease protection.

FIG. 7 illustrates the generation of labeled target sequences by run-off synthesis of labeled RNA using an RNA polymerase.

FIG. 8 illustrates the construction of target sequences indirectly labeled with encoded oligonucleotides that hybridize to differently labeled detection oligonucleotides for implementation of multi-color labeling.

FIG. 9 illustrates the construction of target sequences that are indirectly labeled with a detection oligonucleotide.

FIG. 10 illustrates a scheme for constructing a labeled target sequence by ligating a single strand labeled oligonucleotide.

FIG. 11 illustrates another scheme for constructing a labeled target sequence by ligating a double stranded labeled adaptor.

FIG. 12 illustrates another scheme for constructing a labeled target sequence by ligating a double stranded labeled adaptor.

### DEFINITIONS

Terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Addressable” in reference to tag complements means that the nucleotide sequence, or perhaps other physical or chemical characteristics, of an end-attached probe, such as a tag complement, can be determined from its address, i.e. a one-to-one correspondence between the sequence or other property of the end-attached probe and a spatial location on, or characteristic of, the solid phase support to which it is attached. Preferably, an address of a tag complement is a spatial location, e.g. the planar coordinates of a particular region containing copies of the end-attached probe. However, end-attached probes may be addressed in other ways too, e.g. by microparticle size, shape, color, frequency of micro-transponder, or the like, e.g. Chandler et al, PCT publication WO 97/14028.

“Allele frequency” in reference to a genetic locus, a sequence marker, or the site of a nucleotide means the frequency of occurrence of a sequence or nucleotide at such genetic locus or the frequency of occurrence of such sequence marker, with respect to a population of individuals. In some contexts, an allele frequency may also refer to the frequency of sequences not identical to, or exactly complementary to, a reference sequence.

“Amplicon” means the product of an amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced in a polymerase chain reaction (PCR), by replication in a cloning vector, by linear amplification by an RNA polymerase, such as T7 or SP6, by rolling circle amplification, e.g. Lizardi, U.S. Pat. No. 5,854,033 or Aono et al, Japanese patent publ. JP 4-262799; by whole-genome amplification schemes, e.g. Hosono et al, Genome Research, 13: 959-969 (2003), or by like techniques.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a gene or portion of a gene in a genome, or it may refer to any contiguous portion of genomic sequence whether or not it is within, or associated with, a gene. Preferably, a genetic locus refers to any portion of genomic sequence from a few tens of nucleotides, e.g. 10-30, in length to a few hundred nucleotides, e.g. 100-300, in length.

“Kit” refers to any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3 -29 (1982); and Namsaraev, U.S. patent publication Ser. No. 2004/0110213.

“Microarray” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Typically, the oligonucleotides or polynucleotides are single stranded and are covalently attached to the solid phase support. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999). As used herein, “random microarray” refers to a microarray whose spatially discrete regions of oligonucleotides or polynucleotides are not spatially addressed. That is, the identity of the attached oligonucleoties or polynucleotides is not discernable, at least initially, from its location. Preferably, random microarrays are planar arrays of microbeads wherein each microbead has attached a single kind of hybridization tag complement, such as from a minimally cross-hybridizing set of oligonucleotides. Arrays of microbeads may be formed in a variety of ways, e.g. Brenner et al, Nature Biotechnology, 18: 630-634 (2000); Tulley et al, U.S. Pat. No. 6,133,043; Stuelpnagel et al, U.S. Pat. No. 6,396,995; Chee et al, U.S. Pat. No. 6,544,732; and the like. Likewise, after formation, microbeads, or oligonucleotides thereof, in a random array may be identified in a variety of ways, including by optical labels, e.g. fluorescent dye ratios or quantum dots, shape, sequence analysis, or the like.

“Nucleoside” as used herein includes the natural nucleosides, including 2′-deoxy and 2′-hydroxyl forms, e.g. as described in Kornberg and Baker, DNA Replication, 2 nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structual Biology, 5: 343-355 (1995); and the like. Exemplary types of polynucleotides that are capable of enhancing duplex stability include oligonucleotide N3′→P5′ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Readout” means a parameter, or parameters, which are measured and/or detected that can be converted to a number or value. In some contexts, readout may refer to an actual numerical representation of such collected or recorded data. For example, a readout of fluorescent intensity signals from a microarray is the address and fluorescence intensity of a signal being generated at each hybridization site of the microarray; thus, such a readout may be registered or stored in various ways, for example, as an image of the microarray, as a table of numbers, or the like.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, base-stacking interactions, ionic and hydrophobic interactions, and the like, dominate the interaction of the molecules.

As used herein, the term “Tₘ” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation. Tm=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g., Allawi, H. T. & SantaLucia, J., Jr., Biochemistry 36, 10581-94 (1997)) include alternative methods of computation which take structural and environmental, as well as sequence characteristics into account for the calculation of Tm.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

### DETAILED DESCRIPTION OF THE INVENTION

The present invention provides methods and systems for enhancing signal-to-noise ratios of measurements of labeled target sequences hybridized to complementary sequence attached to solid phase supports, such as microarrays. In one aspect, this objective of the invention is accomplished by generating labeled target sequences that have little or no overhanging ends when hybridized to complementary end-attached probes on the solid phase supports. In another aspect, labeled target sequences are generated by processing amplicons derived from target polynucleotides in a sample or specimen. As explained more fully below, preferably such amplicons are produced using sample-interacting probes that are circularizing probes.

Systems of the invention comprise (i) a set of probes that interact with target polynucleotides in a sample (i.e. “sample-interacting probes”) to produce amplicons that either each contain a segment of a target polynucleotide or an oligonucleotide tag for which there is a predetermined correspondence, usually a one-to-one correspondence, with a particular target polynucleotide or group of target polynucleotides, (ii) one or more solid phase supports that contain a plurality of end-attached probes, and (iii) processing steps wherein the sample-interacting probes of (i) are used to generate amplicons from which labeled target sequences are tailored for the end-attached probes and wherein the resulting labeled target sequences are hybridized to the solid phase supports. In one aspect, the one or more solid phase supports comprises a microarray of end-attached probes. In a preferred embodiment of this aspect, end-attached probe comprise oligonucleotide tags selected from a minimally cross-hybridizing set.

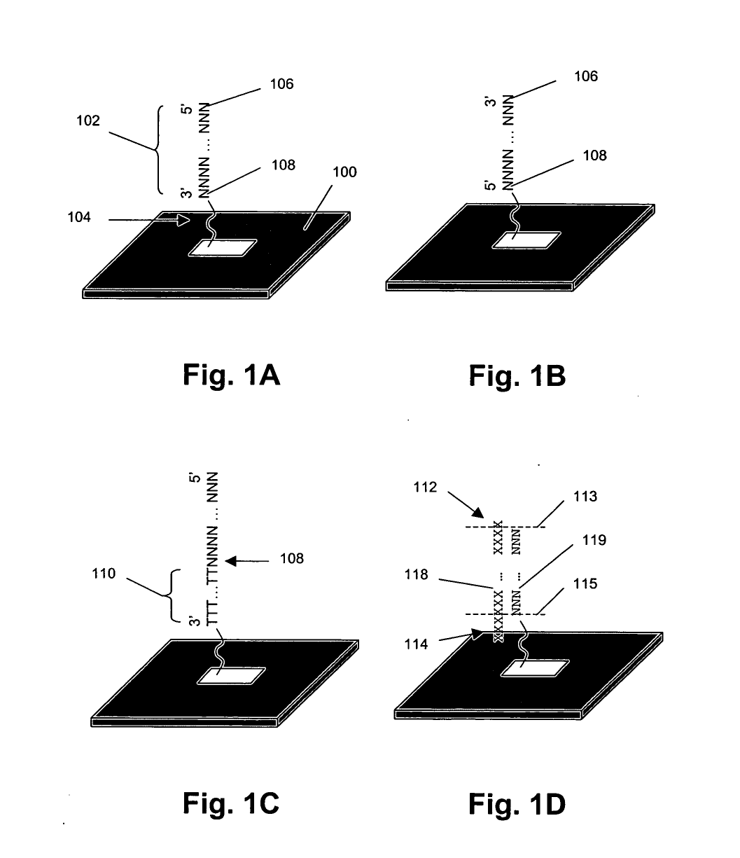

FIGS. 1A-1D illustrate various configuration of end-attached probe on solid phase supports, such as a planar microarray. In FIG. 1A, planar microarray (100) has attached probe (102) to its surface through linker (104) that covalently connects the 3′ carbon of surface-proximal nucleotide (108) to the surface of microarray (100). FIG. 1B illustrates that probe (102) may be attached in the opposite polarity such that a linker covalently connects the 5′ carbon of a surface-proximal nucleotide (108) to the surface of microarray (100). In some case, as illustrated in FIG. 1C, linker (104) may include a sequence of nucleotides (110), which is typically a homopolymeric sequence, such as poly-dT. An important feature of the invention is the degree to which a labeled target sequence (118) overhangs either end an end-attached probe. By way of example, FIG. 1D shows labeled target sequence (118) overhanging the surface-proximal nucleotide of probe (119) by three nucleotides (114) and overhanging the surface-distal nucleotide of probe (119) by one nucleotide (112). Dotted lines (113) and (115) show the ends of probe (119).

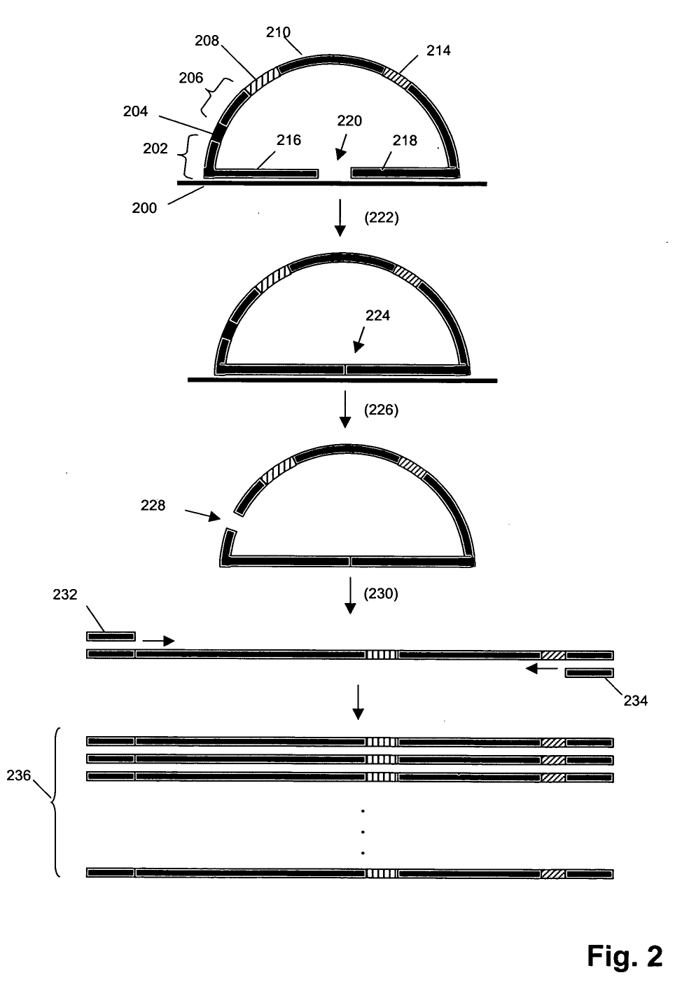

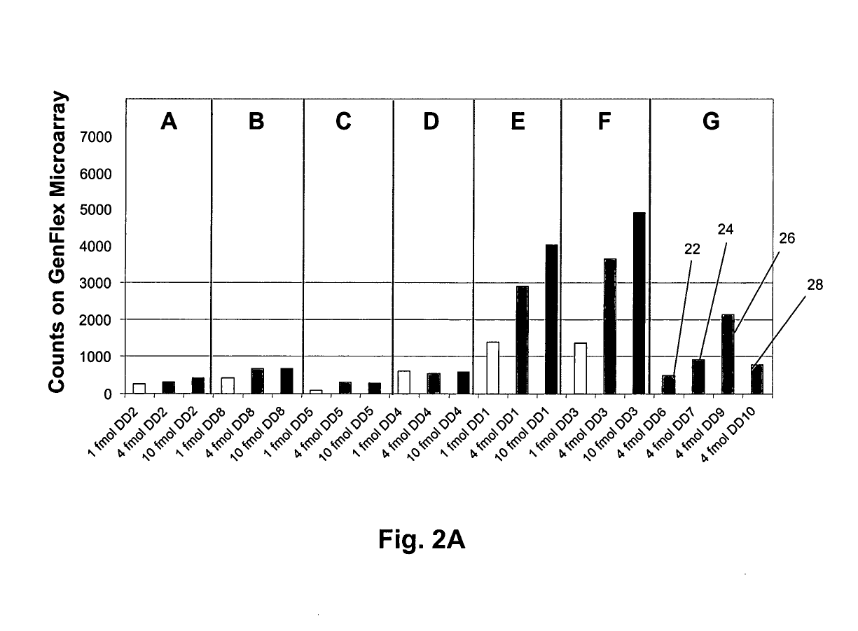

In current practice, the production of labeled target sequences and their application to microarrays leads to degradation in signal-to-noise ratios to a degree roughly proportional to the extent by which the ends of labeled target sequences overhang the ends of their respective probes, as illustrated by the data in FIG. 2A. Ten different fluorescently labeled target sequences were synthesized and applied to a GenFlex microarray (Affymetrix, Santa Clara, Calif.) in the indicated concentrations using the manufacturer's recommended protocols and employing the manufacturer's fluidics station (model FS400). Excitation and signal collection from bound labeled target sequences were carried out with the manufacturer's scanner and data collection instrument. Data analysis was carried out using GeneChip software (Affymetrix). Each of the ten labeled target sequences was design to overhang its complementary end-attached probe by differing amounts, as indicated in the table below. Further, labeled target sequences (DD2, DD8, DD5, and DD4) whose data is shown in panels A-D of FIG. 2A, respectively, have a single fluorescent label attached to the overhang proximal to the GenFlex microarray, i.e. the part of the labeled target sequence overhanging the surface-proximal nucleotide of the end-attached probe. Likewise, label target sequences (DD1, DD3, DD6, DD7, DD9, and DD10) whose data is shown in FIG. 2A in panels E and F, and in bars (22)-(28) of panel G, have a single fluorescent label attached to the overhang distal to the GenFlex microarray, i.e. the part of the labeled target sequence overhanging the surface-distal nucleotide of the end-attached probe.

| | | | | | Approx. |
|---|---|---|---|---|---|
| | Proximal | Distal | Proximal | Distal | Counts |
| Probe | Overhang* | Overhang* | Label | Label | at 4 fmol |
| DD2 | 22 | 0 | YES | NO | 300 |
| DD8 | 5 | 0 | YES | NO | 750 |
| DD5 | 22 | 0 | YES | NO | 350 |
| DD4 | 3 | 0 | YES | NO | 650 |
| DD1 | 0 | 22 | NO | YES | 2950 |
| DD3 | 0 | 0 | NO | YES | 3700 |
| DD6 | 22 | 0 | NO | YES | 500 |
| DD7 | 5 | 0 | NO | YES | 950 |
| DD9 | 0 | 42 | NO | YES | 2100 |
| DD10 | 0 | 42 | NO | YES | 800 |

*Number of nucleotides.

The data show that signal-to-noise ratios of measurements of bound labeled target sequences is higher when the overhang proximal to the surface of the solid phase support is minimized and when the label is not carried on such an overhang.

As mentioned above, labeled target sequences may be generated from samples or specimens using a variety of probes that interact with nucleic acids in the sample or specimen, e.g. usually by the probe containing a segment that specifically hybridizes to a particular complementary target nucleic acid that may serve as ligation and/or extension templates. Such “sample-interacting” probes may include molecular inversion probes, padlock probes, rolling circle probes, ligation-based probes with “zip-code” tags, single-base extension probes, invader probes, and the like, e.g. Hardenbol et al, Nature Biotechnology, 21: 673-678 (2003); Nilsson et al, Science, 265: 2085-2088 (1994); Baner et al, Nucleic Acids Research, 26: 5073-5078 (1998); Lizardi et al, Nat. Genet., 19: 225-232 (1998); Gerry et al, J. Mol. Biol., 292: 251-262 (1999); Fan et al, Genome Research, 10: 853-860 (2000); International patent publications WO 2002/57491 and WO 2000/58516; U.S. Pat. Nos. 6,506,594 and 4,883,750; U.S. Pat. Nos. 5,541,311; 5,614,402; 5,795,763; 6,001,567; and the like, which references are incorporated herein by reference. In one aspect, sample-interacting probes of the invention are circularizing probes, such as padlock probes, rolling circle probes, molecular inversion probes, and the like, e.g. padlock probes being disclosed in U.S. Pat. No. 5,871,921; 6,235,472; 5,866,337; and Japanese patent JP 4-262799; rolling circle probes being disclosed in Aono et al, JP4-262799; Lizardi, U.S. Pat. Nos. 5,854,033; 6,183,960; 6,344,239; and molecular inversion probes being disclosed in Hardenbol et al (cited above) and in Willis et al, U.S. patent publication Ser. No. 2004/0101835, all of which are incorporated herein by reference. Such probes are desirable because non-circularized probes can be digested with single stranded exonucleases thereby greatly reducing background noise due to spurious amplifications, and the like. In the case of molecular inversion probes (MIPs), padlock probes, and rolling circle probes, constructs for generating labeled target sequences are formed by circularizing a linear version of the probe in a template-driven reaction on a target polynucleotide followed by digestion of non-circularized polynucleotides in the reaction mixture, such as target polynucleotides, unligated probe, probe concatatemers, and the like, with an exonuclease, such as exonuclease I.

FIG. 2B illustrates a molecular inversion probe and how it can be used to generate an amplicon after interacting with a target polynucleotide in a sample. A linear version of the probe is combined with a sample containing target polynucleotide (200) under conditions that permit target-specific region 1 (216) and target-specific region 2 (218) to form stable duplexes with complementary regions of target polynucleotide (200). The ends of the target-specific regions may abut one another (being separated by a “nick”) or there may be a gap (220) of several (e.g. 1-10 nucleotides) between them. In either case, after hybridization of the target-specific regions, the ends of the two target specific regions are covalently linked by way of a ligation reaction or an extension reaction followed by a ligation reaction, i.e. a so-called “gap-ligation” reaction. The latter reaction is carried out by extending with a DNA polymerase a free 3′ end of one of the target-specific regions so that the extended end abuts the end of the other target-specific region, which has a 5′ phosphate, or like group, to permit ligation. In one aspect, a molecular inversion probe has a structure as illustrated in FIG. 2B. Besides target-specific regions (216 and 218), in sequence such a probe may include first primer binding site (202), cleavage site (204), second primer binding site (206), first tag-adjacent sequences (208) (usually restriction endonuclease sites and/or primer binding sites) for tailoring one end of a labeled target sequence containing oligonucleotide tag (210), and second tag-adjacent sequences (214) for tailoring the other end of a labeled target sequence. Alternatively, cleavage-site (204) may be added at a later step by amplification using a primer containing such a cleavage site. In operation, after specific hybridization of the target-specific regions and their ligation (222), the reaction mixture is treated with a single stranded exonuclease that preferentially digests all single stranded nucleic acids, except circularized probes. After such treatment, circularized probes are treated (226) with a cleaving agent that cleaves the probe between primer (202) and primer (206) so that the structure is linearized (230). Cleavage site (204) and its corresponding cleaving agent is a design choice for one of ordinary skill in the art. In one aspect, cleavage site (204) is a segment containing a sequence of uracil-containing nucleotides and the cleavage agent is treatment with uracil-DNA glycosylase followed by heating. After the circularized probes are opened, the linear product is amplified, e.g. by PCR using primers (232) and (234), to form amplicons (236). Alternatively to the use of MIPs, amplicons for use with the invention may also be produced as follows. In this method, two universal primer sets are ligated to opposite ends of a target-specific oligonucleotide using the kinetic sampling ligation procedure, e.g. Namsaraev, U.S. patent publication Ser. No. 2004/0110213, which is incorporated herein by reference. The ends of each primer closest to the target-specific oligonucleotides have a short capture sequence, e.g. 6 to 9 nucleotides, preferably 7, which can be from either a random library, e.g. of 7-mers, or a gene-specific set of 7-mers. Each of the two primer sets can contain primers with anywhere from 1 to all possible short-mer capture sequences. After ligation, unligated primers can be removed by such means as exonuclease digestion, if the 5′ end of one primer (C1) and the 3′ end of the other primer (C2) have been suitably protected from such degradation. The ligated products contain only those captured target sequences whose complements were present in the experimental nucleic acid sample. Only these ligation products can be amplified by, for example, PCR using one primer complementary to the constant region, C2, and the original primers (or the C1 sequence alone). After amplification, the appropriate type IIs restriction endonuclease can be used to remove any sequences not found in the queried nucleic acid sample in order to produce target molecules for microarray hybridization which do not have 5′ overhanging sequence (e.g., for 3′-immobilized probe arrays) or 3′ overhanging sequence (e.g., for 5′- immobilized probe arrays). Various labeling methods can be employed including the use of labeled, as discussed below. Reformatting with DNA tags can be accomplished if unique, target-sequence specific short-mer capture sequences are used in the primers. Such DNA tag sequences can be added either 5′ or 3′ to the type IIs r.e. site in either primer (C1 or C2), depending upon the strand and labeling method chosen. This method, too, enables multiplex analysis of nucleic acid samples. Note, also, that if used for genotyping or allele-specific gene expression analysis, strategically positioned mismatches (deletions, etc) either within the target-specific oligo or the primer capture sequences can enhance the specificity of the method. Likewise, the use of LNA, PNA or other modified bases can be employed to enhance the specificity of the target sequence capture event.

### Solid Phase Supports

Solid phase supports for use with the invention may have a wide variety of forms, including planar microarrays, microparticles, beads, bead arrays, and membranes, slides, plates, micromachined chips, and the like. Likewise, solid phase supports of the invention may comprise a wide variety of compositions, including glass, plastic, silicon, alkylthiolate-derivatized gold, cellulose, low cross-linked and high cross-linked polystyrene, silica gel, polyamide, and the like. In one aspect, either a population of discrete particles are employed such that each has a uniform coating, or population, of complementary sequences of the same end-attached probe (and no other), or a single or a few supports are employed with spatially discrete regions each containing a uniform coating, or population, of complementary sequences to the same target sequence (and no other) and distinct from the complementary sequences at the other sites. In the latter embodiment, the area of the regions may vary according to particular applications; usually, the regions range in area from several μm², e.g. 3-5, to several hundred μm², e.g. 100-500. Preferably, such regions are spatially discrete so that signals generated by events, e.g. fluorescent emissions, at adjacent regions can be resolved by the detection system being employed. In some applications, it may be desirable to have regions with uniform coatings of more than one tag complement, e.g. for simultaneous sequence analysis, or for bringing separately tagged molecules into close proximity.

End-attached probes may be used with the solid phase support that they are synthesized on, or they may be separately synthesized and attached to a solid phase support for use, e.g. as disclosed by Lund et al, Nucleic Acids Research, 16: 10861-10880 (1988); Albretsen et al, Anal. Biochem., 189: 40-50 (1990); Wolf et al, Nucleic Acids Research, 15: 2911-2926 (1987); or Ghosh et al, Nucleic Acids Research, 15: 5353-5372 (1987). Preferably, end-attached probes are synthesized on and used with the same solid phase support, which may comprise a variety of forms and include a variety of linking moieties. Such supports may comprise microparticles or microarrays, bead-arrays or matrices. A wide variety of microparticle supports may be used with the invention, including microparticles made of controlled pore glass (CPG), highly cross-linked polystyrene, acrylic copolymers, cellulose, nylon, dextran, latex, polyacrolein, and the like, disclosed in the following exemplary references: Meth. Enzymol., Section A, pages 11-147, vol. 44 (Academic Press, New York, 1976); U.S. Pat. Nos. 4,678,814; 4,413,070; and 4,046;720; and Pon, Chapter 19, in Agrawal, editor, Methods in Molecular Biology, Vol.20, (Humana Press, Totowa, N.J., 1993). Microparticle supports further include commercially available nucleoside-derivatized CPG and polystyrene beads (e.g. available from Applied Biosystems, Foster City, Calif.); derivatized magnetic beads; polystyrene grafted with polyethylene glycol (e.g., TentaGel™, Rapp Polymere, Tubingen Germany); and the like. Selection of the support characteristics, such as material, porosity, size, shape, and the like, and the type of linking moiety employed depends on the conditions under which the end-attached probes are used. For example, in applications involving successive processing with enzymes, supports and linkers that minimize steric hindrance of the enzymes and that facilitate access to substrate are preferred. Other important factors to be considered in selecting the most appropriate microparticle support include size uniformity, efficiency as a synthesis support, degree to which surface area known, and optical properties, e.g. clear smooth beads provide instrumentational advantages when handling large numbers of beads on a surface. Exemplary linking moieties for attaching and/or synthesizing probes on microparticle surfaces are disclosed in Pon et al, Biotechniques, 6:768-775 (1988); Webb, U.S. Pat. No. 4,659,774; Barany et al, International patent application PCT/US91/06103; Brown et al, J. Chem. Soc. Commun., 1989: 891-893; Damha et al, Nucleic Acids Research, 18: 3813-3821 (1990); Beattie et al, Clinical Chemistry, 39: 719-722 (1993); Maskos and Southern, Nucleic Acids Research, 20: 1679-1684 (1992); and the like.

In one aspect, solid phase supports comprising bead populations or bead-arrays are employed as disclosed by Bridgham et al, U.S. Pat. No. 6,406,848; Chandler et al, U.S. Pat. No. 5,981,180; Kettrnan et al, Cytometry, 33: 234-243 (1998); Lerner et al, U.S. Pat. No. 5,716,855; Walt et al, U.S. Pat. No. 6,023,540; Fan et al, Cold Spring Harbor Symposia on Quantitative Biology, 68: 69-78 (2003); which references are incorporated by reference.

In another aspect of the invention, end-attached probes are components of conventional commercially available microarrays, including microfabricated arrays, e.g. as disclosed in Fodor et al, U.S. Pat. Nos. 5,424,186; 5,744,305; 5,445,934; 6,355,432; 6,440,667 (available from Affymetrix, Santa Clara, Calif., particularly the GenFlex product); or as disclosed by Ceirina et al, U.S. Pat. No. 6,375,903 (available from NimbleGen, Madison, Wis.); and “ink-jet” synthesized microarrays, e.g. disclosed in Hughes et al, Nature Biotechnology, 19: 342-347 (2001); Caren et al U.S. Pat. No. 6,323,043, and the like.

End-attached probes may be attached by either a 3′ end or a 5′ end, although for use of high density microarrays, 3 ′-end-attached probes are more readily available commercially. End-attached probes may vary widely in length depending on several factors including whether nucleotide analogs are employed, difficulty of synthesis, number of oligonucleotide tags desired, degree of difference between oligonucleotide tags, and the like. In one aspect, end-attached probes are in the range of from 8 to 60 nucleotides, or from 12 to 50 nucleotides, or from 18 to 40 nucleotides. In accordance with the invention, it is desirable that the lengths of the end-attached probes and the labeled target sequences be substantially identical. “Substantially identical” in this context means that to the extent a labeled target sequence having a single fluorescent label overhangs an end-attached probe, it produces an equivalent signal to that of an equivalent labeled target sequence having no overhangs. Generally, a labeled target sequence overhangs a surface-proximal nucleotide of an end-attached probe by between 0 and 10 nucleotides, or by between 0 and 5 nucleotides, or by between 0 and 2 nucleotides, or preferably by 0 nucleotides. Generally, a labeled target sequence overhangs a surface-distal nucleotide of an end-attached probe by between 0 and 14 nucleotides, or by between 0 and 5 nucleotides, or by between 0 and 2 nucleotides, or preferably by 0 nucleotides. In a further aspect of the invention, labeled target sequences are labeled with one or more fluorescent labels or haptens, such as biotin, digoxigenin, fluorescein, CY5, dinitrophenol, or the like. Preferably, such labels are located at the surface-distal end of a labeled target sequence hybridized to an end-attached probe. More preferaby, such labels are attached to the terminal surface-distal nucleotide of a labeled target sequence hybridized to an end-attached probe.

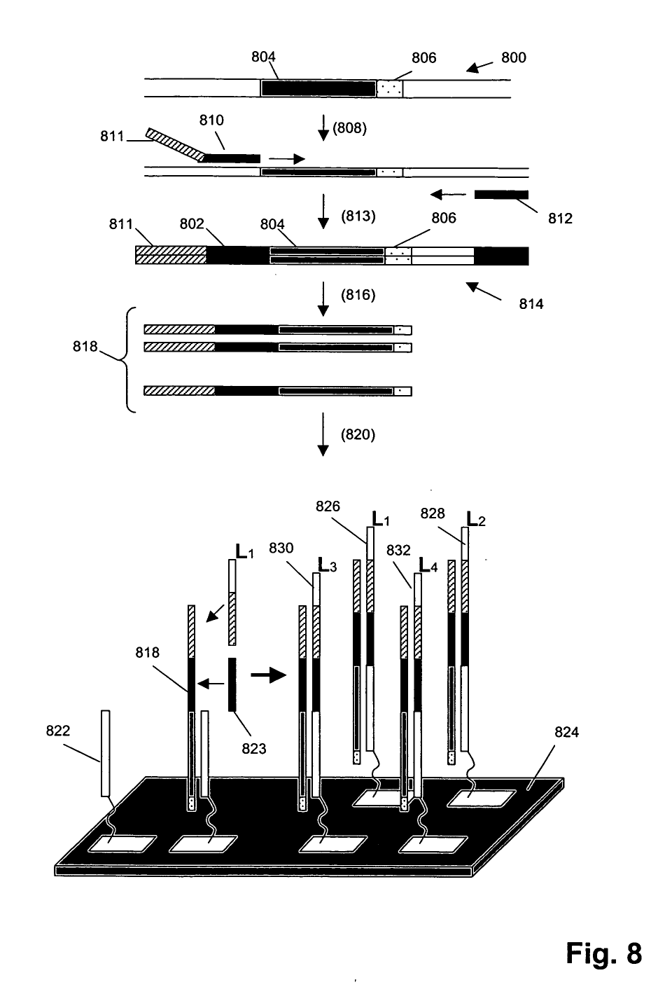

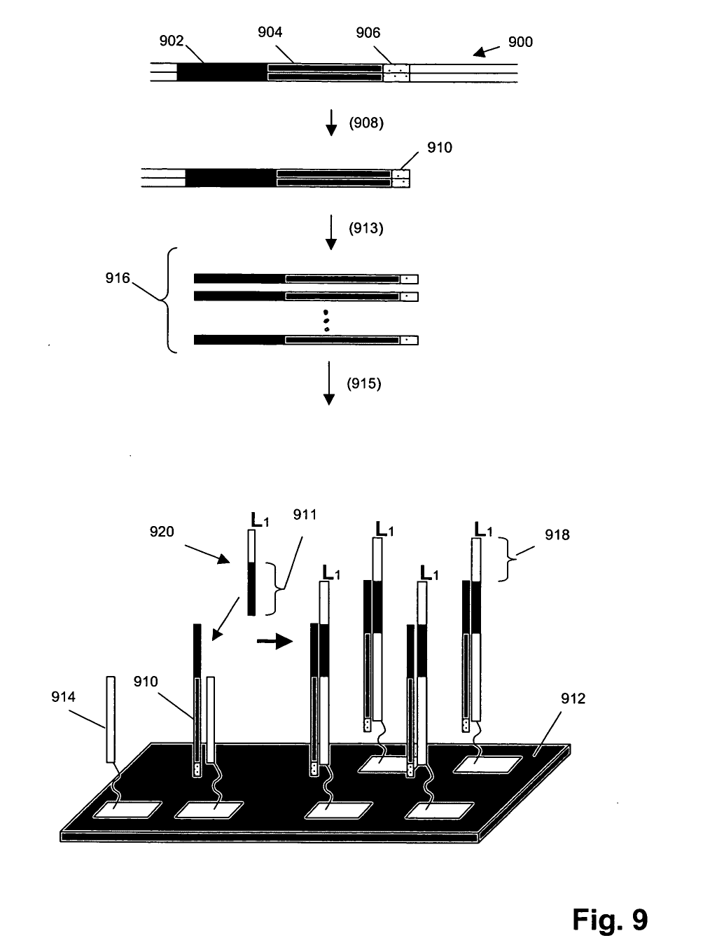

In one aspect of the invention, labeled target sequences are indirectly labeled, as exemplified in FIGS. 8 and 9. In such embodiments, overhangs distal from the surface of a solid phase support are in reference to the end of whatever double-stranded structure is produced in the indirect labeling scheme. For example, in reference to FIG. 9, segment (918) would overhang the surface-distal end of (indirectly) labeled target sequence (910). In such embodiments, segment (911) that detection oligonucleotide (916) hybridizes to may be selected from a minimally cross-hybridizing set. For example, the embodiment of FIG. 8 would employ such a set in order to simultaneously provide four different labels. In one aspect, the size of such a set of minimally cross-hybridizing oligonucleotides is in the range of from 2 to 10, or from 2 to 6, or from 2 to 4.

### Oligonucleotide Tars and Minimally Cross-Hybridizing Sets

In one aspect, the invention provides end-attached probes and labeled target sequences that comprise minimally cross-hybridizing sets of oligonucleotide tags, such as disclosed in Brenner et al, U.S. Pat. No. 5,846,719; Mao et al (cited above); Fan et al, International patent publication WO 2000/058516; Morris et al, U.S. Pat. No. 6,458,530; Morris et al, U.S. patent publication Ser. No. 2003/0104436; Church et al, European patent publication 0 303 459; Huang et al, U.S. Pat. No. 6,709,816; which references are incorporated herein by reference. The sequences of oligonucleotides of a minimally cross-hybridizing set differ from the sequences of every other member of the same set by at least two nucleotides, and more preferably, by at least three nucleotides. Thus, each member of such a set cannot form a duplex (or triplex) with the complement of any other member with less than two mismatches, or three mismatches as the case may be. Preferably, perfectly matched duplexes of tags and tag complements of the same minimally cross-hybridizing set have approximately the same stability, especially as measured by melting temperature. Complements of oligonucleotide tags, referred to herein as “tag complements,” may comprise natural nucleotides or non-natural nucleotide analogs. In one aspect, non-natural nucleic acid analogs are used as tag complements that remain stable under repeated washings and hybridizations of oligonucleoitde tags. In particular, tag complements may comprise peptide nucleic acids (PNAs). Oligonucleotide tags from the same minimally cross-hybridizing set when used with their corresponding tag complements provide a means of enhancing specificity of hybridization. Microarrays of tag complements are available commercially, e.g. GenFlex Tag Array (Affymetrix, Santa Clara, Calif.); and their construction and use are disclosed in Fan et al, International patent publication WO 2000/058516; Morris et al, U.S. Pat. No. 6,458,530; Morris et al, U.S. patent publication Ser. No. 2003/0104436; and Huang et al (cited above).

As mentioned above, in one aspect tag complements comprise PNAs, which may be synthesized using methods disclosed in the art, such as Nielsen and Eghohm (eds.), Peptide Nucleic Acids: Protocols and Applications (Horizon Scientific Press, Wymondham, UK, 1999); Matysiak et al, Biotechniques, 31: 896-904 (2001); Awasthi et al, Comb. Chem. High Throughput Screen., 5: 253-259 (2002); Nielsen et al, U.S. Pat. No. 5,773,571; Nielsen et al, U.S. Pat. No. 5,766,855; Nielsen et al, U.S. Pat. No. 5,736,336; Nielsen et al, U.S. Pat. No. 5,714,331; Nielsen et al, U.S. Pat. No. 5,539,082; and the like, which references are incorporated herein by reference. Construction and use of microarrays comprising PNA tag complements are disclosed in Brandt et al, Nucleic Acids Research, 31(19), e119 (2003).

Preferably, oligonucleotide tags and tag complements are selected to have similar duplex or triplex stabilities to one another so that perfectly matched hybrids have similar or substantially identical melting temperatures. This permits mismatched tag complements to be more readily distinguished from perfectly matched tag complements in the hybridization steps, e.g. by washing under stringent conditions. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Natl. Acad. Sci., 83: 3746-3750 (1986); Wetrnur, Crit. Rev. Biochem. Mol. Biol., 26: 227-259 (1991); and the like. A minimally cross-hybridizing set of oligonucleotides may be screened by additional criteria, such as GC-content, distribution of mismatches, theoretical melting temperature, and the like, to form a subset which is also a minimally cross-hybridizing set.

### Labeled Target Sequences

Labeled target sequences generated in accordance with the invention can be labeled in a variety of ways, including the direct or indirect attachment of fluorescent moieties, colorimetric moieties, chemiluminescent moieties, and the like. Many comprehensive reviews of methodologies for labeling DNA provide guidance applicable to generating labeled oligonucleotide tags of the present invention. Such reviews include Haugland, Handbook of Fluorescent Probes and Research Chemicals, Ninth Edition (Molecular Probes, Inc., Eugene, 2002); Keller and Manak, DNA Probes, 2 nd Edition (Stockton Press, New York, 1993); Eckstein, editor, Oligonucleotides and Analogues: A Practical Approach (IRL Press, Oxford, 1991); Wetmur, Critical Reviews in Biochemistry and Molecular Biology, 26: 227-259 (1991); and the like. Particular methodologies applicable to the invention are disclosed in the following sample of references: Fung et al, U.S. Pat. No. 4,757,141; Hobbs, Jr., et al U.S. Pat. No. 5,151,507; Cruickshank, U.S. Pat. No. 5,091,519. In one aspect, one or more fluorescent dyes are used as labels for labeled target sequences, e.g. as disclosed by Menchen et al, U.S. Pat. No. 5,188,934 (4,7-dichlorofluorscein dyes); Begot et al, U.S. Pat. No. 5,366,860 (spectrally resolvable rhodamine dyes); Lee et al, U.S. Pat. No. 5, 847,162 (4,7-dichlororhodamine dyes); Khanna et al, U.S. Pat. No. 4,318,846 (ether-substituted fluorescein dyes); Lee et al, U.S. Pat. No. 5,800,996 (energy transfer dyes); Lee et al, U.S. Pat. No. 5,066,580 (xanthene dyes): Mathies et al, U.S. Pat. No. 5,688,648 (energy transfer dyes); and the like. Labeling can also be carried out with quantum dots, as disclosed in the following patents and patent publications, incorporated herein by reference: U.S. Pat. Nos. 6,322,901; 6,576,291; 6,423,551; 6,251,303; 6,319,426; 6,426,513; 6,444,143; 5,990,479; 6,207,392; Ser. Nos. 2002/0045045; 2003/0017264; and the like. As used herein, the term “fluorescent signal generating moiety” means a signaling means which conveys information through the fluorescent absorption and/or emission properties of one or more molecules. Such fluorescent properties include fluorescence intensity, fluorescence life time, emission spectrum characteristics, energy transfer, and the like.

Commercially available fluorescent nucleotide analogues readily incorporated into the labeling oligonucleotides include, for example, Cy3-dCTP, Cy3-dUTP, Cy5-dCTP, Cy5-dUTP (Amersham Biosciences, Piscataway, N.J., USA), fluorescein-12-dUTP, tetramethylrhodamine-6-dUTP, Texas Red®-5-dUTP, Cascade Blue®-7-dUTP, BODIPY® FL-14-dUTP, BODIPY®R-14-dUTP, BODIPY® TR-14-dUTP, Rhodamine Green™-5-dUTP, Oregon Green® 488-5-dUTP, Texas Red®-12-dUTP, BODIPY® 630/650-14-dUTP, BODIPY® 650/665-14-dUTP, Alexa Fluor® 488-5-dUTP, Alexa Fluor® 532-5-dUTP, Alexa Fluor® 568-5-dUTP, Alexa Fluor® 594-5-dUTP, Alexa Fluor® 546-14-dUTP, fluorescein-12-UTP, tetramethylrhodamine-6-UTP, Texas Red®-5-UTP, Cascade Blue®-7-UTP, BODIPY® FL-14-UTP, BODIPY® TMR-14-UTP, BODIPY® TR-14-UTP, Rhodanine Green™-5-UTP, Alexa Fluor® 488-5-UTP, Alexa Fluor® 546-14-UTP (Molecular Probes, Inc. Eugene, Oreg., USA). Protocols are available for custom synthesis of nucleotides having other fluorophores. Henegariu et al., “Custom Fluorescent-Nucleotide Synthesis as an Alterative Method for Nucleic Acid Labeling,” Nature Biotechnol. 18:345-348 (2000), the disclosure of which is incorporated herein by reference in its entirety.

Other fluorophores available for post-synthetic attachment include, inter alia, Alexa Fluor® 350, Alexa Fluor® 532, Alexa Fluor® 546, Alexa Fluor® 568, Alexa Fluor® 594, Alexa Fluor® 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Texas Red (available from Molecular Probes, Inc., Eugene, Oreg., USA), and Cy2, Cy3.5, Cy5.5, and Cy7 (Amersham Biosciences, Piscataway, N.J. USA, and others).

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver particles may be coated onto the surface of the array to enhance signal from fluorescently labeled oligos bound to the array. Lakowicz et al., BioTechniques 34: 62-68 (2003).

The label may instead be a radionucleotide, such as ³³P, ³²P, ³⁵S, and ³H.

Biotin, or a derivative thereof, may also be used as a label on a detection oligonucleotide, and subsequently bound by a detectably labeled avidin/streptavidin derivative (e.g. phycoerythrin-conjugated streptavidin), or a detectably labeled anti-biotin antibody. Digoxigenin may be incorporated as a label and subsequently bound by a detectably labeled anti-digoxigenin antibody (e.g. fluoresceinated anti-digoxigenin). An aminoallyl-dUTP residue may be incorporated into a detection oligonucleotide and subsequently coupled to an N-hydroxy succinimide (NHS) derivitized fluorescent dye, such as those listed supra. In general, any member of a conjugate pair may be incorporated into a detection oligonucleotide provided that a detectably labeled conjugate partner can be bound to permit detection. As used herein, the term antibody refers to an antibody molecule of any class, or any subfragment thereof, such as an Fab.

Other suitable labels for detection oligonucleotides may include fluorescein (FAM), digoxigenin, dinitrophenol (DNP), dansyl, biotin, bromodeoxyuridine (BrdU), hexahistidine (6×His), phosphor-amino acids (e.g. P-tyr, P-ser, P-thr) , or any other suitable label. In one embodiment the following hapten/antibody pairs are used for detection, in which each of the antibodies is derivatized with a detectable label: biotin/α-biotin, digoxigenin/α-digoxigenin, dinitrophenol (DNP)/α-DNP, 5-Carboxyfluorescein (FAM)/α-FAM.

As described in schemes below, target sequences may also be indirectly labeled, especially with a hapten that is then bound by a capture agent, e.g. as disclosed in Holtke et al, U.S. Pat. Nos. 5,344,757; 5,702,888; and 5,354,657; Huber et al, U.S. Pat. No. 5,198,537; Miyoshi, U.S. Pat. No. 4,849,336; Misiura and Gait, PCT publication WO 91/17160; and the like. Many different hapten-capture agent pairs are available for use with the invention, either with a target sequence or with a detection oligonucleotide used with a target sequence, as described below. Exemplary, haptens include, biotin, des-biotin and other derivatives, dinitrophenol, dansyl, fluorescein, CY5, and other dyes, digoxigenin, and the like. For biotin, a capture agent may be avidin, streptavidin, or antibodies. Antibodies may be used as capture agents for the other haptens (many dye-antibody pairs being commercially available, e.g. Molecular Probes).

### Schemes for Generating Labeled Target Sequences

Labeled target sequences within the scope of the invention may be formed and labeled in a variety of ways as exemplified below and as may be further designed by one of ordinary skill with reference to the present teaching. In the examples below, the usual starting point is an amplicon or cDNA library containing either portions of target sequences or oligonucleotide tags that have a well-defined, usually one-to-one, correspondence with target sequences. In one aspect, such oligonucleotide tags are from a minimally cross-hybridizing set.

The schemes below are implemented using conventional molecular biology techniques well known to those of ordinary skill in the art, as exemplified by references such as Sambrook et al, Molecular Cloning: A Laboratory Manual, Second Edition (Cold Spring Harbor Laboratory Press, New York, 1989) and Brent et al, editors, Current Protocols in Molecular Biology (John Wiley & Sons, New York, 2003), from which protocols set forth below are incorporated by reference. In the schemes described below, one of ordinary skill in the art recognizes that the placement of the various elements in amplicons, such as primer binding sites, restriction sites, and the like, are carried out so that after cleavage, or amplification, or labeling, the resulting labeled target sequences are in accordance with the invention.

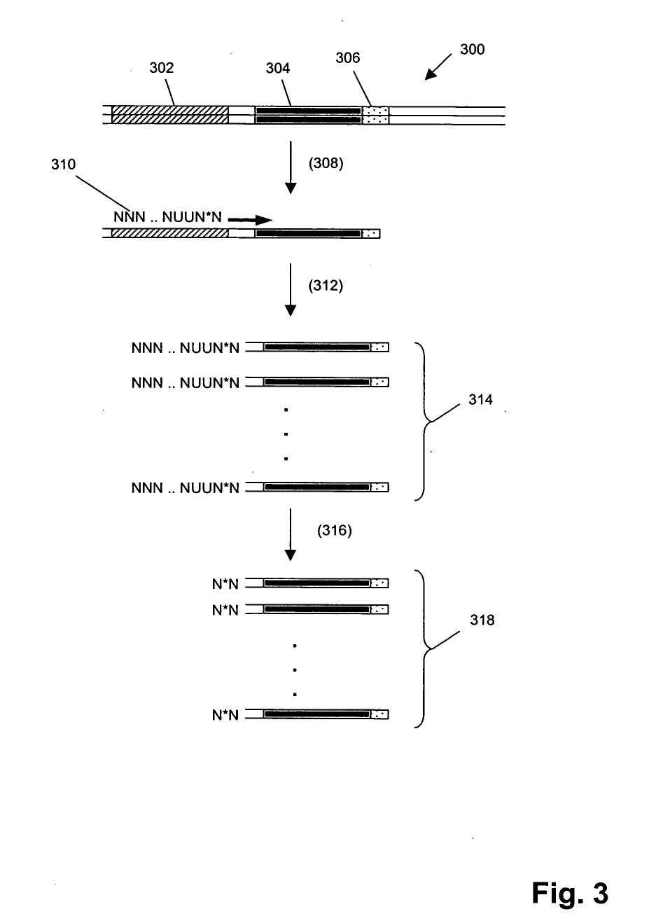

FIG. 3 illustrates one approach for construction of labeled target sequences from amplicons, e.g. generated from molecular inversion probes. Amplicon (300) has in sequence primer binding site (302), target sequence (304), which for example may be an oligonucleotide tag of a molecular inversion probe, and restriction endonuclease site (306), which may be a type II restriction endonuclease, such as DraI, or a type IIs restriction endonuclease positioned to cleave amplicon (300) at the boundary of target sequence (304). Amplicon (300) is cleaved (308) with a restriction endonuclease that recognizes site (306) to remove downstream sequence from target sequence (304). The resulting product is denatured and primer (310) is added to the reaction mixture under conditions that allow it to anneal to the complementary strand of primer binding site (302). Primer (310) is constructed to contain one or more deoxyuridines on the 5′-side of a labeled nucleotide, indicated by “N*” in the figure. A DNA polymerase and the appropriate dNTP substrates are added to the reaction mixture to extend (312) primer (310) to copy a strand of target sequence (304) so that structure (314) is formed. Optionally, successive cycles of denaturation, annealing, and extension may be employed to increase the amount of label target sequence eventually produced. In any case, uracil-DNA glycosylase is added (316) to the reaction mixture to remove the uracils from the nucleosides of primer (310), after which primer (310) is cleaved at those sites by heating or by addition of an apurinic/apyrimidinic (AP) endonuclease to give labeled target sequence (318). Optionally, labeled target sequence (318) may be purified using conventional techniques before application to end-attached probes on solid phase supports. Uracil-DNA glycosylase and AP endonuclease are readily available commercially (e.g. New England Biolabs, Beverly, Mass.) and may be used in accordance with the manufacturer's suggested protocols. Alternatively, deoxyuridines may be replaced with a riboNTP and the sequences cleaved with base (e.g. NaOH) and heat. In yet another embodiment, prior to restriction digestion, similarly designed cleavable primers may be used in exponential PCR, in conjunction with a 2^{nd} downstream primer, to create labeled amplicons which are then digested with a restriction endonuclease and UNG (for example) to give labeled targets of similar structure (318) suitable for chip hybridization. In still another embodiment, a Type IIS restriction endonuclease site embedded in the labeling primer, may be used to cleave away undesired DNA 5′ of the primer's labeling moiety.

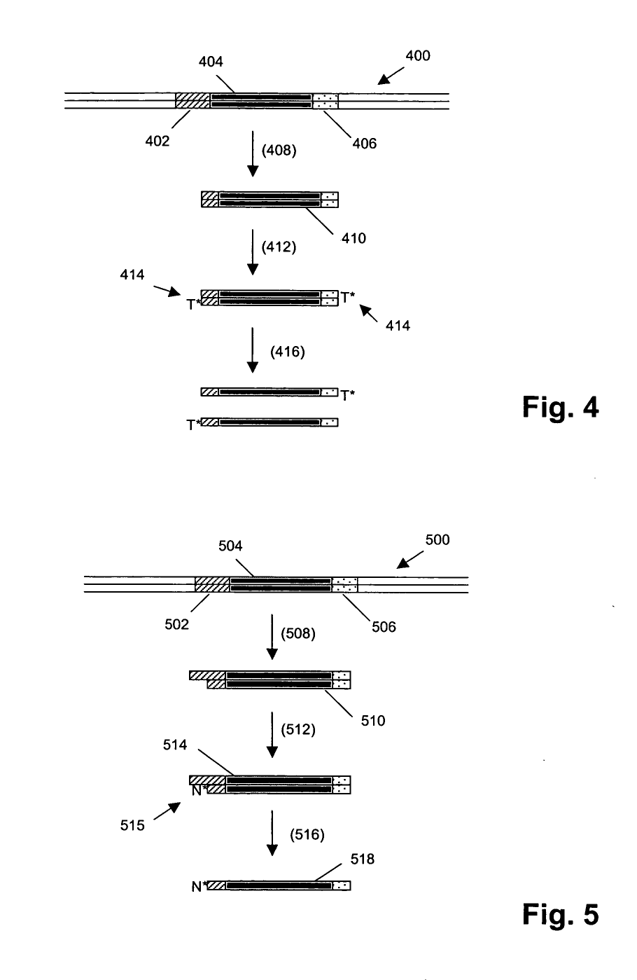

FIG. 4 illustrates another scheme for constructing labeled target sequences using terminal transferase labeling. Amplicon (400) has target sequences (404) that are flanked by restriction endonuclease sites (402) and (406), which may be the same or different, or may be for type II or type IIs restriction endonucleases. Amplicon (400) is cleaved (408) with the restriction endonucleases recognizing sites (402) and (406) to give structure (410), which is then labeled (412) at the 3′ end of each strand by addition of a labeled dideoxynucleotide using a terminal transferase. The resulting labeled fragment (414) is then denatured (416) and optionally purified to give labeled target sequences that may be specifically hybridized to end-attached probes of a solid phase support, such as a microarray.

FIG. 5 illustrates another scheme for constructing labeled target sequences by polymerase extension of target sequences with one or more labeled nucleotides. Amplicon (500) has target sequence (504) that is flanked by restriction endonuclease cleavage site (502), that upon cleavage results in fragments having 5′ overhangs, and endonuclease cleavage site (506) that preferably leaves a blunt end or a 3′ overhang to prevent labeling of the “upper” strand. In one aspect, site (502) is the cleavage site of a type IIs restriction endonuclease, which allows the nucleotide sequence of the cleavage site to be a design choice. Suitable type IIs restriction endonucleases leaving 5′ overhangs include SapI and AlwI, which are commercially available (e.g. New England Biolabs, Beverly, Mass.). Both sites (502) and (506) are cleaved (508) giving fragment (510) from which labeled fragment (514) is formed, after extension by a DNA polymerase in the presence of appropriate dNTPs, including one or more labeled dNTPs. Labeled fragments (514) are denatured to produce labeled target sequences for application to a microarray, or the like.

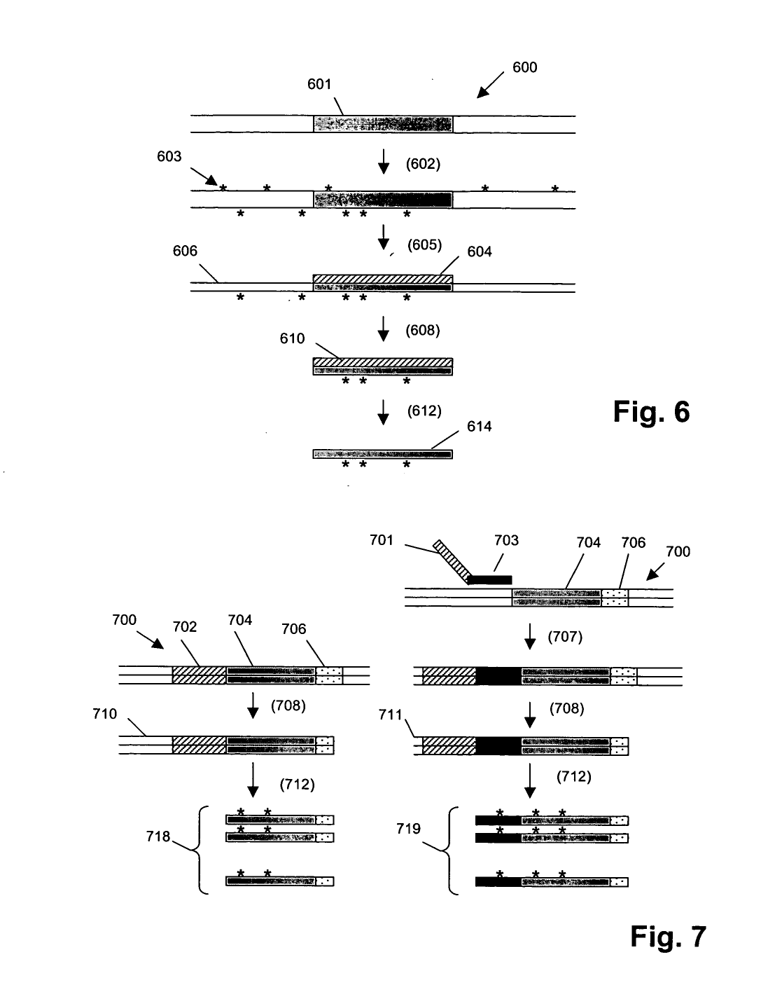

FIG. 6 illustrates another scheme for constructing labeled target sequences by protecting a region of a full length labeled target sequence from digestion by a single-stranded exonuclease, such as exonuclease I or S1 nuclease. Labeled amplicon (603) is formed by PCR (602) of amplicon (600) in the presence of one or more labeled dNTPs, or by nick translation in the presence of one or more labeled dNTPs, or by like labeling technique. Asterisks (*) indicate an exemplary distribution of labeled nucleotides in amplicon (603). After denaturing (605) amplicon (603), protection oligonucleotide (604) is hybridized to labeled strand (606) of denatured amplicon (603). Protection oligonucleotide (604) is selected to be exactly complementary to labeled target sequences within amplicon (603). Whenever oligonucleotide tags are employed, protection oligonucleotides (604) have the same sequences as the end-attached probes. After a duplex is formed between strand (606) and protection oligonucleotide (604), a single stranded exonuclease is added (608) under conditions that permit the digestion of the single strands overhanging protection oligonucleotide (604) to give labeled duplex (610). Labeled duplex (610) is then denatured (612) to free labeled target sequence (614) for application to end-attached probes on a solid phase support. Essentially the same procedure may be followed using protection oligonucleotides that are labeled. Protection oligonucleotides failing to form duplexes with target sequences in denatured amplicons are digested; the surviving labeled protection oligonucleotide are then used as labeled target sequences.

FIG. 7 illustrates schemes for constructing labeled target sequences using an RNA polymerase. In one case, promoter (702) is inserted into amplicon (700), and in the other case, promoter site (701) is added in a PCR reaction using primer (703). In the first case, amplicon (700) contains target sequence (704) that is flanked by promoter (702) for an RNA polymerase and restriction endonuclease site (706). Suitable RNA polymerases include T7 and SP6 RNA polymerases, which are readily available commercially (e.g. New England Biolabs, Beverly, Mass.). After digestion (708) of amplicon (700) with a restriction endonuclease recognizing site (706), resulting fragments (710) are combined (712) with an appropriate RNA polymerase in the presence of one or more labeled NTPs to form labeled target sequences (718). After labeled target sequences are separated from the labeled NTPs, they may be applied to end-attached probes on a microarray, or like support. In the other case, after generating (707) an amplicon containing promoter (701), it is cleaved (708) with a restriction endonuclease recognizing site (706) to give fragment (711), to which is added an RNA polymerase and NTPs to generated labeled target sequences (719).

FIG. 8 illustrates a scheme for multi-color labeling using labeled target sequences that are indirectly labeled via encoded oligonucleotides that are each encoded to specifically hybridize to one of a plurality of detection oligonucleotides. The detection oligonucleotides are then labeled with a fluorophor or a hapten or other signal generating moiety. Multi-color labeling may be advantageous in schemes to detect single-nucleotide polymorphisms (SNPs) or transcript levels from multiple samples using molecular inversion probes, padlock probes, rolling circle probes, or the like. For example, as described above, in the application of molecular inversion probes to detect SNPs, four reactions are carried out in different reaction vessels to separately generate circularized probes for each of four possible nucleotides that might occupy a specific site of a test sequence. Thus, amplicon (800) may be one of a set of four amplicons that are processed to produce differently labeled target sequences. In each case, a resulting amplicon (800) contains target sequence (804) flanked by primer binding site (802) and restriction endonuclease recognition site (806). Amplicon (800) is further amplified with primers (810) and (812). Primer (810) contains an encoding segment (811) that may be an oligonucleotide selected from a minimally cross-hybridizing set. After amplification, resulting product (814) is formed that contains in sequence: encoding segment (811), primer binding site (802), target sequence (804), and restriction site (806). After digestion with a restriction endonuclease that recognizes site (806), the resulting fragment is denatured (816) to give target sequence (818), that is indirectly labeled with encoded segment (811). Indirectly labeled target sequence (818) may be specifically hybridized to end-attached probes (822) on solid phase support (824). Target sequences are labeled by specifically hybridizing to the microarry a mixture of four directly labeled detection oligonucleotides (826-832, labeled with labels “L₁” through “L₄” respectively), each containing a complement of one of four encoded segments (811). At the same time, an additional oligonucleotide (823), referred to herein as a “filler oligonucleotide,” is specifically hybridized to the region of the detection oligonucleotide that is complementary to primer (810). Thus, three contiguous oligonucleotides are specifically hybridized to the labeled target sequence: an end-attached probe, a filler oligonucleotide, and a detection oligonucleotide. This configuration increases the stability of the complex by base-stacking. In alternative embodiments, there may be a plurality of filler oligonucleotides, either in a linear end-to-end configuration, or they may be overlapping and complementary to one another. Filler oligonucleotide may be labeled or unlabeled.

FIG. 9 illustrates a scheme for single-color indirect labeling of target sequences. Amplicon (900) contains target sequence (904) flanked by primer binding site (902) and restriction endonuclease recognition site (906). After digestion (908) with a restriction endonuclease that recognizes site (906), fragment (910) is formed, which is denatured (913) to form indirectly labeled target sequences (916). Indirectly labeled target sequences (916) are specifically hybridized to end-attached probes (914) on solid phase support (912). Finally, labeled detection oligonucleotide (920) containing a segment (911) complementary to a strand of primer binding site (902) is specifically hybridized to its complement on labeled target sequence (910).

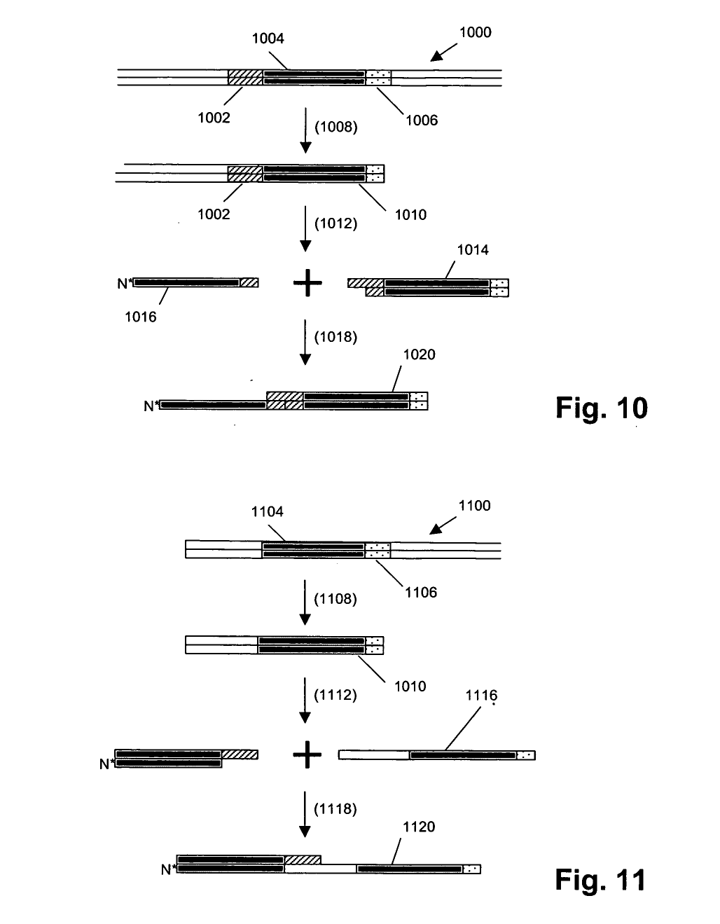

FIG. 10 illustrates a scheme for constructing a labeled target sequence by ligating a single strand labeled oligonucleotide. Amplicon (1000) contains target sequence (1004) flanked by first restriction endonuclease site (1002) and second restriction endonuclease site (1006) ), the latter preferably leaving a blunt end after digestion. First restriction endonuclease recognizing site (1002) is selected so that it leaves a 5′ overhang upon digestion. After digestion (1008) with second restriction endonuclease recognizing site (1006), fragment (1010) is generated, which is then digested (1012) with the first restriction endonuclease to give fragment (1014). To fragment (1014) is added a 3′-labeled, 5′-phosphorylated oligonucleotide (1016) whose 5′ end is complementary to the overhang of fragment (1014). After annealing and ligation (1018), labeled fragment (1020) is formed, which is denatured and hybridized to a solid phase support.

FIG. 11 illustrates another scheme for constructing a labeled target sequence by ligating a double stranded labeled adaptor. Amplicon (1100) contains target sequence (1104) flanked by restriction endonuclease site (1006). After cleavage (1108) with restriction endonuclease recognizing site (1106), fragment (1110) is formed. Fragment (1110) is denatured (1112) to give single strand (1116), which is mixed with labeled adaptor (1114). Labeled adaptor (1114) has a label on the 3′ end of one strand and at the opposite end it has an overhanging 3′ end whose sequence is complementary to the 3′ end of single strand (1116). Adaptor (1114) and single strand (1116) are incubated together under ligation conditions (1118) so that labeled double stranded fragment (1020) is formed, which may be denatured and hybridized to a solid phase support.

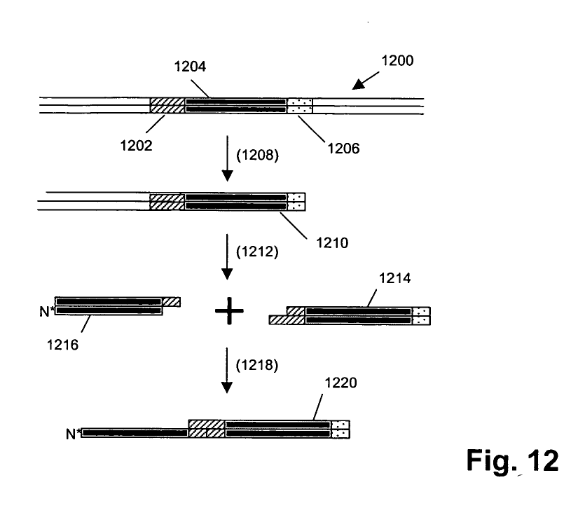

FIG. 12 illustrates another scheme for constructing a labeled target sequence by ligating a double stranded labeled adaptor. Amplicon (1200) contains target sequence (1204) flanked by first restriction endonuclease site (1202) and second restriction endonuclease site (1206). First restriction endonuclease recognizing site (1202) is selected so that it leaves a 5′ overhang upon digestion. After digestion (1208) with second restriction endonuclease recognizing site (1206), preferably leaving a blunt end, fragment (1210) is generated, which is then digested (1212) with the first restriction endonuclease to give fragment (1214). To fragment (1214) is added a 3′-labeled, 5′-phosphorylated adaptor (1216) whose 5′ end is complementary to the overhang of fragment (1214). After annealing and ligation (1218), labeled fragment (1220) is formed, which is denatured and hybridized to a solid phase support.

### Hybridization of Labeled Target Sequence to Solid Phase Supports

Methods for hybridizing labeled target sequences to microarrays, and like platforms, suitable for the present invention are well known in the art. Guidance for selecting conditions and materials for applying labeled target sequences to solid phase supports, such as microarrays, may be found in the literature, e.g. Wetmur, Crit. Rev. Biochem. Mol. Biol., 26: 227-259 (1991); DeRisi et al, Science, 278: 680-686 (1997); Chee et al, Science, 274: 610-614 (1996); Duggan et al, Nature Genetics, 21: 10-14 (1999); Schena, Editor, Microarrays: A Practical Approach (IRL Press, Washington, 2000); Freeman et al, Biotechniques, 29: 1042-1055 (2000); and like references. Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference. Hybridization conditions typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will stably hybridize to a perfectly complementary target sequence, but will not stably hybridize to sequences that have one or more mismatches. The stringency of hybridization conditions depends on several factors, such as probe sequence, probe length, temperature, salt concentration, concentration of organic solvents, such as formamide, and the like. How such factors are selected is usually a matter of design choice to one of ordinary skill in the art for any particular embodiment. Usually, stringent conditions are selected to be about 5° C. lower than the Tₘ for the specific sequence for particular ionic strength and pH. Exemplary hybridization conditions include salt concentration of at least 0.01 M to no more than 1 M Na ion concentration (or other salts) at a pH 7.0 to 8.3 and a temperature of at least 25° C. Additional exemplary hybridization conditions include the following: 5×SSPE (750 mM NaCl, 50 mM sodium phosphate, 5 mM EDTA, pH 7.4).

Exemplary hybridization procedures for applying labeled target sequence to a GenFlex™ microarray (Affymetrix, Santa Clara, Calif.) is as follows: denatured labeled target sequence at 95-100° C. for 10 minutes and snap cool on ice for 2-5 minutes. The microarray is pre-hybridized with 6×SSPE-T (0.9 M NaCl 60 mM NaH₂,PO₄, 6 mM EDTA (pH 7.4),0.005% Triton X-100) +0.5 mg/ml of BSA for a few minutes, then hybridized with 120 μL hybridization solution (as described below) at 42° C. for 2 hours on a rotisserie, at 40 RPM. Hybridization Solution consists of 3M TMACL (Tetramethylanmmonium. Chloride), 50 mM MES ((2-[N-Morpholino]ethanesulfonic acid) Sodium Salt) (pH 6.7), 0.01% of Triton X-100, 0.1 mg/ml of Herring Sperm DNA, optionally 50 pM of fluorescein-labeled control oligonucleotide, 0.5 mg/ml of BSA (Sigma) and labeled target sequences in a total reaction volume of about 120 μL. The microarray is rinsed twice with 1×SSPE-T for about 10 seconds at room temperature, then washed with 1×SSPE-T for 15-20 minutes at 40° C. on a rotisserie, at 40 RPM. The microarray is then washed 10 times with 6×SSPE-T at 22° C. on a fluidic station (e.g. model FS400, Affymetrix, Santa Clara, Calif.). Further processing steps may be required depending on the nature of the label(s) employed, e.g. direct or indirect. Microarrays containing labeled target sequences may be scanned on a confocal scanner (such as available commercially from Affymetrix) with a resolution of 60-70 pixels per feature and filters and other settings as appropriate for the labels employed. GeneChip Software (Affymetrix) may be used to convert the image files into digitized files for further data analysis.

### Detection of Hybridized Labeled Target Sequences

Labeled target sequences of the invention are detected by specifically hybridizing them to one or more solid supports containing end-attached probes, usually in the form of a microarray of spatially discrete hybridization sites. Instruments for measuring optical signals, especially fluorescent signals, from labeled tags hybridized to targets on a microarray are described in the following references which are incorporated by reference: Stem et al, PCT publication WO 95/22058; Resnick et al, U.S. Pat. No. 4,125,828; Karnaukhov et al, U.S. Pat. No. ,354,114; Trulson et al, U.S. Pat. No. 5,578,832; Pallas et al, PCT publication WO 98/53300; and the like.

The above teachings are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such changes and modifications that fall within the true spirit and scope of the invention.

1. A method of enhancing signal-to-noise ratios of measurements from one or more solid phase supports having end-attached probes, the method comprising the steps of:
providing one or more solid phase supports, each having a surface and one or more end-attached probes, each of such probes having a surface-proximal end nucleotide, a surface-distal end nucleotide, and a nucleotide sequence;
providing labeled target sequences from a sample such that (i) each labeled target sequence comprises a first end nucleotide, a second end nucleotide, and a nucleotide sequence complementary to the nucleotide sequence of at least one end-attached probe of a solid phase support, and (ii) in duplexes formed between labeled target sequences and end-attached probes, the first end nucleotide of each labeled target sequence overhangs the surface-proximal nucleotide of the end-attached probe by from 0 to 10 nucleotides and the second end nucleotide of each labeled target sequence overhangs the surface-distal nucleotide of the end-attached probe by from 0 to 14 nucleotides; and
mixing under hybridizing conditions labeled target sequences with the one or more solid phase supports so that duplexes form between labeled target sequences and end-attached, and so that the labels of the labeled target sequences generate signals from the one or more solid phase supports.

2. The method of claim 1 wherein said labeled target sequences are each labeled with one or more light-generating molecules for producing optical signals or with one or more hapten molecules that may be combined with capture agents for producing optical signals, the optical signals indicating the presence of a labeled target sequence at an end-attached probe.

3. The method of claim 2 wherein said one or more solid phase supports is a microarray or a random microarray each having a plurality of said end-attached probes.

4. The method of claim 3 wherein said labeled target sequences comprises a set of minimally cross-hybridizing oligonucleotide tags and said end-attached probes on said microarray or said random microarray comprise a set of tag complements of such minimally cross-hybridizing oligonucleotides.

5. The method of claim 4 wherein said plurality of said end-attached probes is a number between 50 and 100,000, and wherein each of said plurality of said end-attached probes has a length in the range of from eight to sixty nucleotides.

6. The method of claim 5 wherein said plurality of said end-attached probes is a number between 100 and 50,000

7. The method of claim 4 wherein said duplexes formed between said labeled target sequences and said end-attached probes, said first end nucleotide of each of said labeled target sequence overhangs said surface-proximal nucleotide of said end-attached probe by from 0 to 5 nucleotides and said second end nucleotide of each of said labeled target sequence overhangs said surface-distal nucleotide of said end-attached probe by from 0 to 5 nucleotides.

8. The method of claim 7 wherein said duplexes formed between said labeled target sequences and said end-attached probes, said first end nucleotide of each of said labeled target sequences overhangs said surface-proximal nucleotide of said end-attached probe by from 0 to 2 nucleotides and said second end nucleotide of each of said labeled target sequence overhangs said surface-distal nucleotide of said end-attached probe by from 0 to 2 nucleotides.

9. The method of claim 8 wherein said first end nucleotide of each of said labeled target sequences is base-paired with said surface-proximal nucleotide of said end-attached probe.

10. The method of claim 9 wherein said step of providing said labeled target sequences includes forming an amplicon by amplifying a target sequence from a sample-interacting probe.

11. The method of claim 10 wherein said sample-interaction probe is a circularizing probe that has been converted into a covalently closed circle by a template-driven ligation reaction between the circularizing probe and a target nucleic acid in a sample.

12. The method of claim 11 wherein said circularizing probe is selected from the group consisting of molecular inversion probes, padlock probes, and rolling circle probes.

13. The method of claim 12 wherein said circularizing probe is a molecular inversion probe and said amplicon is formed by linearizing the molecular inversion probe and amplifying said target sequence by a polymerase chain reaction.

14. The method of claim 13 wherein said labeled target sequence is formed by (i) providing a 3′-end-labeled primer specific for a strand of said amplicon, the 3 ′-end-labeled primer containing one or more uracil bases; (ii) annealing and extending with a DNA polymerase the 3′end-labeled primer on said amplicon to form a labeled primer-target sequence conjugate; and (iii) treating the 3′-end-labeled primer-target sequence conjugate with uracil-DNA-glycolsylase to cleave said primer at the uracils, thereby forming a 5′-end-labeled target sequence.

15. The method or claim 13 wherein said labeled target sequence is formed by (i) providing restriction endonuclease sites flanking said target sequence in said amplicon, (ii) digesting said amplicon with restriction endonucleases recognizing such sites to form a target sequence fragment having 3′ ends, and (iii) labeling the 3′ ends of the target sequence fragment with a terminal transferase in the presence of a dideoxynucleoside triphosphate, thereby forming a 3′-end-labeled target sequence.

16. The method of claim 13 wherein said labeled target sequence is formed by (i) providing a first restriction endonuclease site recognized by a first restriction endonuclease that cleaves such site to leave a 5′ overhang and a second restriction endonuclease site recognized by a second restriction endonuclease that cleaves such site to leave a blunt end or a 3′ overhang, the first and second restriction endonuclease sites flanking said target sequence in said amplicon, (ii) digesting said amplicon with the first and second restriction endonucleases to form a target sequence fragment having a 3′-recessed end, and (iii) labeling the 3′-recessed end of the target sequence fragment by extending such end with a DNA polymerase in the presence of a labeled terminator, thereby forming a 3′-end-labeled target sequence.

17. The method of claim 13 wherein said labeled target sequence is formed by (i) providing a labeled amplicon by amplifying said amplicon in a polymerase chain reaction that includes one or more labeled deoxynucleoside triphosphates, (ii) denaturing the labeled amplicon, (iii) annealing a protection oligonucleotide to said target sequence of the labeled amplicon to form a protected duplex, and (iv) treating the protected duplex with a single-stranded exonuclease, thereby forming said labeled target sequence.

18. The method of claim 13 wherein said labeled target sequence is formed by (i) providing a promoter site and restriction site flanking said target sequence in said amplicon, (ii) digesting said amplicon with a restriction endonuclease recognizing the restriction site to form a target sequence fragment, and (iii) treating the target sequence fragment with an RNA polymerase recognizing the promoter in the presence of one or more labeled ribonucleoside triphosphates so that labeled oligoribonucleotides are synthesized to provide a labeled target sequence.

19. The method of claim 13 wherein said labeled target sequence is formed by (i) providing a first restriction endonuclease site recognized by a first restriction endonuclease that cleaves such site to leave a 5′ overhang and a second restriction endonuclease site recognized by a second restriction endonuclease that cleaves such site to leave a blunt end or a 3′ overhang, the first and second restriction endonuclease sites flanking said target sequence in said amplicon, (ii) digesting said amplicon with the first and second restriction endonucleases to form a target sequence fragment having a 5′ overhang, and (iii) labeling the 3′-recessed end of the target sequence fragment by ligating to such end a 3′-labeled 5′-phosphorylated oligonucleotide having a complementary end to the 5′ overhang, thereby forming a 3′-end-labeled target sequence.

20. A method of enhancing signal-to-noise ratios of measurements from one or more solid phase supports, each having end-attached probes, the method comprising the steps of:
providing one or more solid phase supports, each having a surface and one or more end-attached probes, each of such probes having a surface-proximal end nucleotide, a surface-distal end nucleotide, and a nucleotide sequence;
providing labeled target sequences from a sample, each labeled target sequence comprising (i) a first segment having a first end nucleotide and a nucleotide sequence complementary to the nucleotide sequence of at least one end-attached and (ii) a second segment having a predetermined sequence having a length in the range of from 8 to 60 nucleotides, the second segment overhanging the surface-distal nucleotide of the end-attached probe whenever a duplex is formed between a labeled target sequence and such end-attached probe;
providing for each second segment one or more detection oligonucleotides, each having an end complementary to the predetermined sequence of the second segment of at least one labeled target sequence such that the end of at least one of the one or more detection oligonucleotides abuts the surface-distal nucleotide of the end-attached probe, at least one detection oligonucleotide being labeled with one or more light-generating molecules for producing optical signals or with one or more hapten molecules that may be combined with capture agents for producing optical signals; and
mixing under hybridizing conditions the labeled target sequences and the detection oligonucleotides with the one or more solid phase supports so that duplexes form between labeled target sequences and end-attached probes and between the second segment of labeled target sequences and detection oligonucleotides and so that the labels of the detection oligonucleotides generate signals from the one or more solid phase supports.

21. The method of claim 20 wherein said one or more solid phase supports is a microarray or a random microarray each having a plurality of said end-attached probes.

22. The method of claim 21 wherein said labeled target sequences comprises a set of minimally cross-hybridizing oligonucleotide tags and said end-attached probes on said microarray or said random microarray comprise a set of tag complements of such minimally cross-hybridizing oligonucleotides.

23. (Canceled)

23. The method of claim 22 wherein said plurality of said end-attached probes is a number between 50 and 100,000, and wherein each of said plurality of said end-attached probes has a length in the range of from eight to sixty nucleotides.

24. The method of claim 23 wherein said plurality of said end-attached probes is a number between 100 and 50,000

25. The method of claim 24 wherein in said duplexes formed between said labeled target sequences and said end-attached probes, said first end nucleotide of each of said labeled target sequences overhangs said surface-proximal nucleotide of said end-attached probe by from 0 to 5.

26. The method of claim 25 wherein in said duplexes formed between said labeled target sequences and said end-attached probes, said first end nucleotide of each of said labeled target sequences is base-paired with said surface-proximal nucleotide of said end-attached.

27. The method of claim 26 wherein said step of providing said labeled target sequences includes forming an amplicon by amplifying a target sequence from a sample-interacting probe.

28. The method of claim 27 wherein said sample-interaction probe is a circularizing probe that has been converted into a covalently closed circle by a template-driven ligation reaction between the circularizing probe and a target nucleic acid in a sample.

29. The method of claim 28 wherein said circularizing probe is selected from the group consisting of molecular inversion probes, padlock probes, and rolling circle probes.

30. The method of claim 29 wherein said circularizing probe is a molecular inversion probe and said amplicon is formed by linearizing the molecular inversion probe and amplifying said target sequence by a polymerase chain reaction.

31. A system for providing a multiplex readouts for genetic measurements on a sample, the system comprising:
a set of sample-interacting probes that interact with target polynucleotides in a sample to produce amplicons that either each contain a segment of a target polynucleotide or an oligonucleotide tag for which there is a predetermined correspondence with a particular target polynucleotide or group of target polynucleotides; and
one or more solid phase supports having a plurality of end-attached probes, each end-attached probe having a surface-proximal nucleotide and a surface-distal oligonucleotide;
wherein labeled target sequences are generated from the amplicons so that each labeled target sequence overhangs the surface-proximal nucleotide of a complementary end-attached probe by a number of nucleotide in the range of from 0 to 10 and the surface-distal nucleotide of a complementary end-attached probe by a number of nucleotide in the range of from 0 to 14 whenever a duplex is formed therebetween.

32. The system of claim 31 wherein each said labeled target sequence overhangs said surface-proximal nucleotide of said complementary end-attached probe by a number of nucleotide in the range of from 0 to 5 and said surface-distal nucleotide of said complementary end-attached probe by a number of nucleotide in the range of from 0 to 5 whenever a duplex is formed therebetween.

33. The system of claim 32 wherein said one or more solid phase supports is a microarray or a random microarray each having a plurality of said end-attached probes, and wherein said labeled target sequences comprises a set of minimally cross-hybridizing oligonucleotide tags and said end-attached probes on said microarray or said random microarray comprise a set of tag complements of such minimally cross-hybridizing oligonucleotides.

34. The system of claim 33 wherein said sample-interaction probe is a circularizing probe that has been converted into a covalently closed circle by a template-driven ligation reaction between the circularizing probe and a target nucleic acid in said sample.

35. The system of claim 34 wherein said circularizing probe is a molecular inversion probe.

36. The system of claim 35 wherein each said labeled target sequence overhangs said surface-proximal nucleotide of said complementary end-attached probe by a number of nucleotide in the range of from 0 to 2 and said surface-distal nucleotide of said complementary end-attached probe by a number of nucleotide in the range of from 0 to 2 whenever a duplex is formed therebetween.

37. The method of claim 22 where said one or more detection oligonucleotides includes at least one filler oligonucleotide.
